(19) United States
(12) Patent Application Publication
(10) Pub. No.: US 2026/0345995 A1
(43) Pub. Date: Nov. 5, 2026

(54) DYNAMICALLY DEACTIVATING LAYERS OF A MACHINE LEARNING MODEL TO DETECT PHYSIOLOGICAL SIGNALS

(71) Applicant: Medtronic, Inc., Minneapolis, MN (US)

(72) Inventors: Kristin N. Hageman, Dayton, MN (US); Joshua J. Nedrud, Roseville, MN (US); Joel A. Anderson, Brooklyn Park, MN (US); Thaddeus S. Brink, St. Paul, MN (US); Philip E. Tracton, Porter Ranch, CA (US)

(21) Appl. No.: 19/659,307

(22) Filed: Apr. 27, 2026

Related U.S. Application Data
(60) Provisional application No. 63/799,081, filed on May 2, 2025.

(51) Int. Cl. A61N 1/02; A61N 1/36; G16H 40/67

(52) U.S. Cl. CPC A61N 1/025; A61N 1/36139; G16H 40/67

(57) ABSTRACT

An example implantable medical device includes a storage medium configured to store a machine learning model including a first set of layers, a second set of layers, and a third set of layers; and processing circuitry configured to receive first physiological data and second physiological data from sensing circuitry; apply the first physiological data to the first set of layers to generate a first base output; apply the first base output to the second set of layers to generate a first detection output; determine, based on the first detection output, to deactivate the second set of layers; in response to the determination to deactivate the second set of layers: deactivate the second set of layers; apply the second physiological data to the first set of layers to generate a second base output; and apply the second base output to the third set of layers to generate a second detection output.

### CROSS-REFERENCE TO RELATED APPLICATION

This application claims the benefit of and priority to U.S. Provisional Patent Application No. 63/799,081 filed May 2, 2025, the entire disclosure of which is incorporated by reference herein.

### FIELD

The disclosure relates generally to medical device systems and, more particularly, medical device systems configured to detect and analyze physiological signals.

### BACKGROUND

Medical devices may be used to monitor physiological signals of a patient. For example, some medical devices are configured to sense cardiac electrogram (EGM) signals indicative of the electrical activity of the heart via electrodes, electroencephalogram (EEG) signals indicative of the electrical activity of the brain via electrodes, and/or an evoked compound action potential (ECAP). Some medical devices may be configured to deliver a therapy in conjunction with or separate from the monitoring of physiological signals.

### SUMMARY

A medical device, such as an implantable medical device (IMD), may be configured to sense one or more physiological signals of a patent. Example physiological signals may include at least one of an electroencephalogram (EEG) signal, electrocardiogram (ECG) signal, signals that vary based on respiration, signals that vary based on blood pressure, signals that vary based on activity level, signals that vary based on posture, signals that vary based on sleep, signals that vary based on medication state, electromyography (EMG) signal, evoked potential, evoked compound action potential (ECAP), intrinsic nerve activity, local field potential signal, and/or electrophysiology (EP). These signals may be sensed using one or more sensors, such as one or more electrodes, accelerometers, or any other sensors.

As described herein, a system may modify the application of portions of a machine learning (ML) model to improve efficiency and/or speed of signal analysis using the ML model. For example, a particular physiological signal, such as an ECAP signal, may be more easily detectable in sensed physiological data and can be determined via a shallow set of layers of a ML model. In other patients, the particular physiological signal, such as an ECAP signal, may not be as easily detectable in sensed physiological data and may require a more computationally expensive set of layers to determine whether a particular signal, such as an ECAP signal, is present in the sensed physiological data in the particular patient.

In accordance with techniques described herein, a medical device, such as an IMD, may dynamically deactivate a set of layers in a ML model to save computational resources and improve efficiency of the ML model. For example, an IMD may dynamically deactivate a particular set of layers of the ML model (e.g., a shallow set of layers) based on whether an initial output of the particular layers satisfies a deactivation threshold.

In some examples, an IMD may sense physiological data, such as physiological signals, and processing circuitry of IMD may input physiological data sensed during an initial period of time to a first set of layers in a ML model to generate a first base output, and then apply the first base output to a second set of layers of the ML model to determine generate a first detection output. In some examples, the first detection output may be an indication of whether a particular signal, such as an ECAP, EEG signal, ECG signal, is detected in the physiological data. Processing circuitry of IMD may determine whether to deactivate the second set of layers for additional physiological data, such as physiological data sensed after the physiological data sensed during an initial period of time, based on the first detection output. For example, processing circuitry of IMD may determine whether the first detection output satisfies a deactivation threshold. In some examples, the first detection output satisfying the deactivation threshold may correspond to the first detection output, which is output by the second set of layers of the ML model, indicating a physiological signal, such as an ECAP signal, ECG signal, or EEG signal, is not detected in the first physiological data (e.g., the second set of layers of the ML model was unable to identify a satisfactory desired particular signal, such as an ECAP, in the physiological data).

In some examples, in response to a determination the first detection output satisfies the deactivation threshold, processing circuitry of IMD may determine to deactivate the second set of layers of the ML model. In some examples, in response to a determination the first detection output satisfies the deactivation threshold, processing circuitry may additionally determine to activate a third set of layers of the ML model, where executing the third set of layers of the ML model is more computationally expensive than executing the second set of layers of the ML model, but the third set of layers, while more computationally expensive, may be configured to more accurately detect particular signal in physiological data than the second set of layers of the ML model.

In response to a determination to deactivate the second set of layers of the ML model, processing circuitry of IMD is configured to apply second physiological data to the first set of layers of the ML model to generate a second base output and is configured to apply the second base output to the third set of layers of the ML model, while skipping the deactivated second set of layers of the ML model, to generate a second output detection, such as an indication of whether a particular signal, such as an ECAP, EEG signal, ECG signal, is detected in the additional physiological data.

In one example, this disclosure describes an implantable medical device comprising: sensing circuitry configured to generate first physiological data representative of first physiological signals of a patient and second physiological data representative of second physiological signals of the patient, the second physiological signals being sensed after the first physiological signals; a storage medium configured to store a machine learning (ML) model comprising a plurality of layers, the plurality of layers comprising a first set of layers, a second set of layers, and a third set of layers; and processing circuitry configured to: receive the first physiological data and the second physiological data from the sensing circuitry; apply the first physiological data to the first set of layers to generate a first base output; apply the first base output to the second set of layers to generate a first detection output; determine, based on the first detection output, to deactivate the second set of layers for the second physiological data; in response to the determination to deactivate the second set of layers: deactivate the second set of layers; apply the second physiological data to the first set of layers to generate a second base output; and apply the second base output to the third set of layers to generate a second detection output.

In another example, this disclosure describes a method comprising: generating, by sensing circuitry of an implantable medical device (IMD), first physiological data representative of first physiological signals of a patient and second physiological data representative of second physiological signals of the patient; applying, by processing circuitry of the IMD, the first physiological data to a first set of layers of a machine learning (ML) model to generate a first base output; applying, by the processing circuitry, the first base output to a second set of layers of the ML model to generate a first detection output; determining, by the processing circuitry and based on the first detection output, to deactivate the second set of layers for the second physiological data based on the first detection output; in response to the determination to deactivate the second set of layers: deactivating, by the processing circuitry, the second set of layers; applying, by the processing circuitry, the second physiological data to the first set of layers to generate a second base output; and applying, by the processing circuitry, the second base output to a third set of layers of the ML model to generate a second detection output.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A illustrates the environment of an example medical system in conjunction with a patient, in accordance with one or more techniques disclosed herein.

FIG. 1B illustrates the environment of an example medical system in conjunction with a patient, in accordance with one or more techniques disclosed herein.

FIG. 2 is a functional block diagram illustrating an example configuration of the implantable medical device (IMD) of the medical system of FIGS. 1A or 1B in accordance with one or more techniques disclosed herein.

FIGS. 3A, 3B, and 3C are conceptual diagrams illustrating example processes for a machine learning model, in accordance with one or more techniques disclosed herein.

FIG. 4 is a functional block diagram illustrating an example configuration of the external device of FIGS. 1A-1B, in accordance with one or more techniques disclosed herein.

FIG. 5 is a block diagram illustrating an example system that includes an access point, a network, external computing devices, such as a server, and one or more other computing devices, which may be coupled to the IMD and external device of FIGS. 1–4, in accordance with one or more techniques disclosed herein.

FIG. 6 is a flow diagram illustrating an example technique for determining whether to deactivate a set of layers of a machine learning model, in accordance with one or more techniques disclosed herein.

Like reference characters denote like elements throughout the description and figures.

### DETAILED DESCRIPTION

A variety of types of implantable medical devices (IMDs) sense physiological signals that include physiological data, such as cardiac EGMs, EEGs, or ECAPs. In some examples, EGMs may also include electrocardiogram (ECGs or EKGs). In some examples, electrodes used by IMDs to sense physiological signals are typically integrated with a housing of the IMD and/or coupled to the IMD via one or more elongated leads. Example IMDs that monitor physiological data and/or provide therapy include spinal cord stimulators, deep brain stimulation devices, sacral nerve or tibial nerve stimulation devices, and pacemakers and implantable cardioverter-defibrillators. These devices may be coupled to intravascular and/or extravascular leads, as well as include with housings configured for implantation within the heart or other anatomical location that are leadless (e.g., carry electrodes on the housing). An example of pacemaker configured for intracardiac implantation is the Micra™ Transcatheter Pacing System, available from Medtronic plc. Some IMDs that do not provide therapy, e.g., implantable patient monitors, sense cardiac EGMs. Examples of such an IMD are the Reveal LINQ™ and LINQ II™ Insertable Cardiac Monitors, available from Medtronic plc, which may be inserted subcutaneously. Such IMDs may facilitate relatively longer-term monitoring of patients during normal daily activities and may periodically transmit collected data to a network service, such as the Medtronic Carelink™ Network.

In some examples, an IMD attempting to sense a particular signal may also sense noise, and on some occasions the amount of noise included in the sensed signal may make the desired sensed signal, such as an ECAP, EEG signal, or ECG signal, more difficult to identify in a signal sensed by the IMD. In some examples, an IMD may input one or more of the sensed parameters or features derived from the sensed parameters into a machine learning (ML) model to detect a presence of a particular signal, such as an ECAP, EEG signal, or ECG signal, in the sensed signal. In some examples, an IMD may input one or more of the sensed parameters or features derived from the sensed parameters into a ML model to determine one or more physiological parameters, diagnostic parameters, and/or therapeutic parameters.

An IMD may have limited battery and/or limited memory size for an IMD to be small enough to be effectively implantable in a patient. When a ML model is operating on an IMD, computational resources and/or power may be limited due to size limitations of an IMD. The power and/or memory needs for running ML models are often high, which may be problematic for an IMD to run one or more ML models due to the limited battery and/or the limited memory size of an IMD.

In accordance with techniques described herein, processing circuitry of an IMD may be configured to dynamically deactivate a particular set of layers in a ML model (e.g., a shallow set of layers) based on an initial detection output of the particular set of layers which may save computational resources and improve efficiency of ML model by not executing the particular sets of layers that are not generating useful output.

An IMD having a ML model, in accordance with techniques described herein, that is configured to operate with improved efficiency may enable the IMD to have a greater lifespan and/or enable the IMD to run the ML model more often, which may help produce improved clinical results for a patient. In some examples, having one ML model that dynamically deactivates particular sets of layers to determine whether a particular signal is detected in sensed physiological data instead of multiple separate ML models may save computational resources, such as memory, of an IMD.

In some patients, a particular physiological signal, such as an ECAP signal, may be more easily detectable in sensed physiological data and can be determined via a shallow output layer set of a ML model. In other patients, the particular physiological signal, such as an ECAP signal, may not be as easily detectable in sensed physiological data and may require a more computationally expensive set of layers to determine whether a particular signal, such as an ECAP signal, is present in the sensed physiological data in the particular patient.

Any medical device configured to sense physiological data via implanted electrodes or sensors, including the examples identified herein, may implement the techniques of this disclosure for applying sensed physiological data to particular sets of layers of a ML model to determine which sets of layers of the ML model to activate or deactivate to analyze additional sensed physiological data.

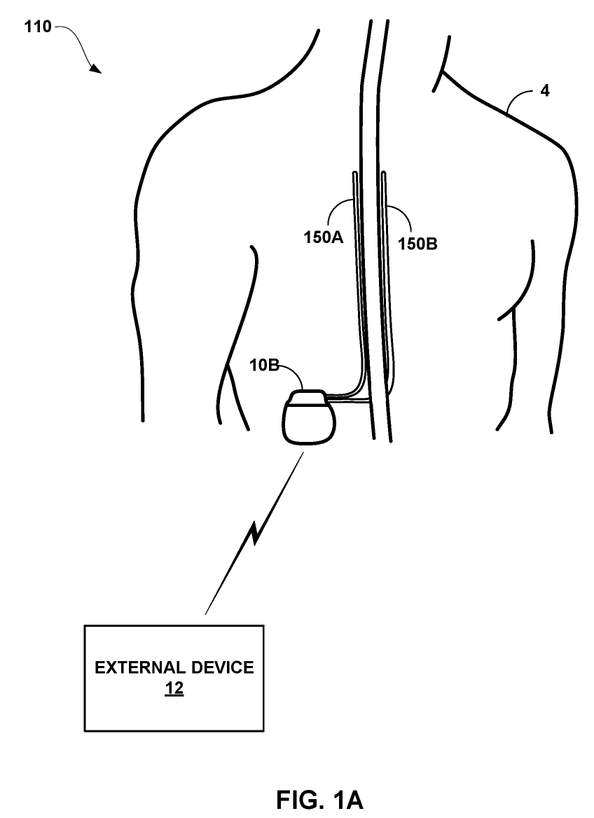

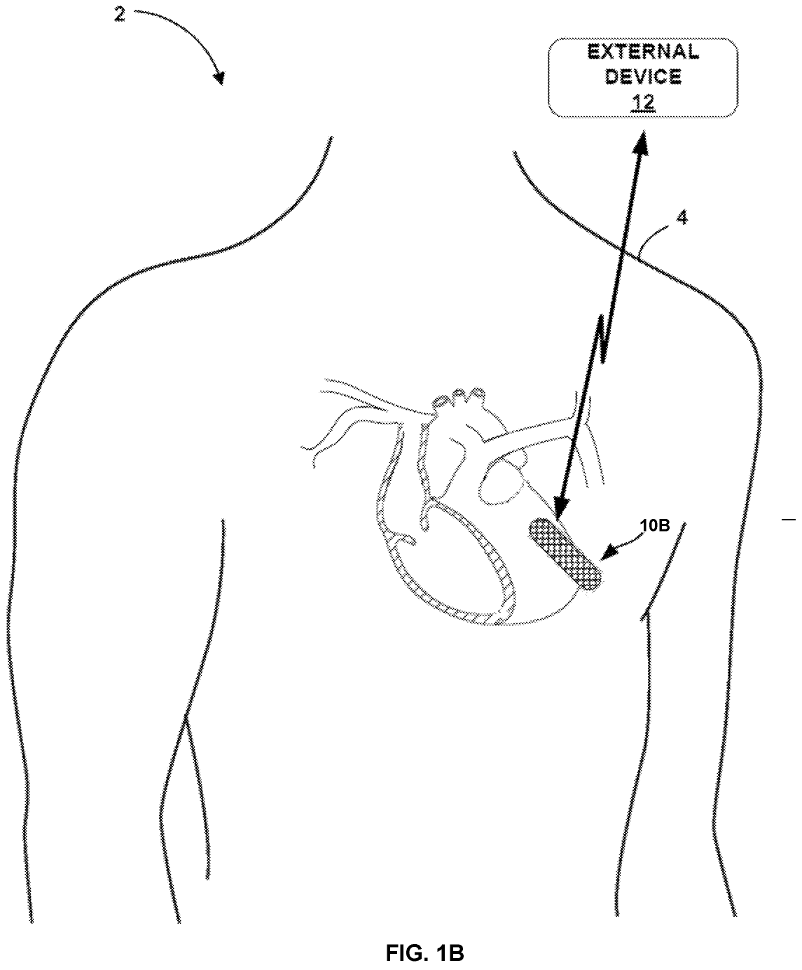

FIG. 1A illustrates a conceptual diagram illustrating an example system 110 that includes another example of an IMD 10A that is configured to deliver spinal cord stimulation (SCS) therapy and an external device 12, in accordance with one or more techniques of this disclosure.

Although the techniques described in this disclosure are generally applicable to a variety of IMDs, application of such techniques to IMDs and, more particularly, an ICM or implantable electrical stimulators (e.g., neurostimulators) will be described for purposes of illustration. More particularly, the disclosure will refer to an IMD 10A system or implantable SCS system for purposes of illustration, but without limitation as to other types of medical devices or other therapeutic applications of medical devices.

As shown in FIG. 1A, system 110 includes an IMD 10A, leads 150A and 150B, and external programmer 150 shown in conjunction with a patient 4, who is ordinarily a human patient. In the example of FIG. 1, IMD 10A is an implantable electrical stimulator that is configured to generate and deliver electrical stimulation therapy to patient 4 via one or more electrodes of electrodes of leads 150A and/or 150B (collectively, “leads 150”), e.g., for relief of chronic pain or other symptoms. In other examples, IMD 10A may be coupled to a single lead carrying multiple electrodes or more than two leads each carrying multiple electrodes. In some examples, the stimulation signals, or pulses, may be configured to elicit detectable ECAP signals that are sensed by IMD 10A. In some examples, IMD 10A may use sensed ECAP signals to determine a posture state occupied by patient 4 and/or determine how to adjust one or more parameters that define stimulation therapy. In this manner, IMD 10A may sense one or more physiological signals and use one or more characteristics of those signals as feedback for closed-loop control of the electrical stimulation that is delivered. IMD 10A may be a chronic electrical stimulator that remains implanted within patient 4 for weeks, months, or even years. In other examples, IMD 10A may be a temporary, or trial, stimulator used to screen or evaluate the efficacy of electrical stimulation for chronic therapy. In one example, IMD 10A is implanted within patient 4, while in another example, IMD 10A is an external device coupled to percutaneously implanted leads. In some examples, IMD 10A uses one or more leads, while in other examples, IMD 10A is leadless.

FIG. 1B illustrates the environment of another example medical system 2 in conjunction with a patient 4, in accordance with one or more techniques of this disclosure. The example techniques may be used with an IMD 10B, which may be in wireless communication with at least one of external device 12 and other devices not pictured in FIG. 1. In some examples, IMD 10B is implanted outside of a thoracic cavity of patient 4 (e.g., subcutaneously in the pectoral location illustrated in FIG. 1). IMD 10B may be positioned near the sternum near or just below the level of the heart of patient 4, e.g., at least partially within the cardiac silhouette. IMD 10B includes a plurality of electrodes (not shown in FIG. 1) and is configured to sense a cardiac EGM via the plurality of electrodes. In some examples, IMD 10A takes the form of the Reveal LINQ™ or LINQ II™ ICM, or another ICM similar to, e.g., a version or modification of, the Reveal LINQ™ or LINQ II™ ICMs.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, personal digital assistant, or another computing device that may run an application that enables the computing device to interact with IMD 10A or IMD 10B. External device 12 is configured to communicate with IMD 10A or IMD 10B and, optionally, another computing device (not illustrated in FIG. 1), via wireless communication. External device 12, for example, may communicate via near-field communication technologies (e.g., inductive coupling, NFC or other communication technologies operable at ranges less than 10–20cm) and far-field communication technologies (e.g., RF telemetry according to the 802.11 or Bluetooth® specification sets, or other communication technologies operable at ranges greater than near-field communication technologies).

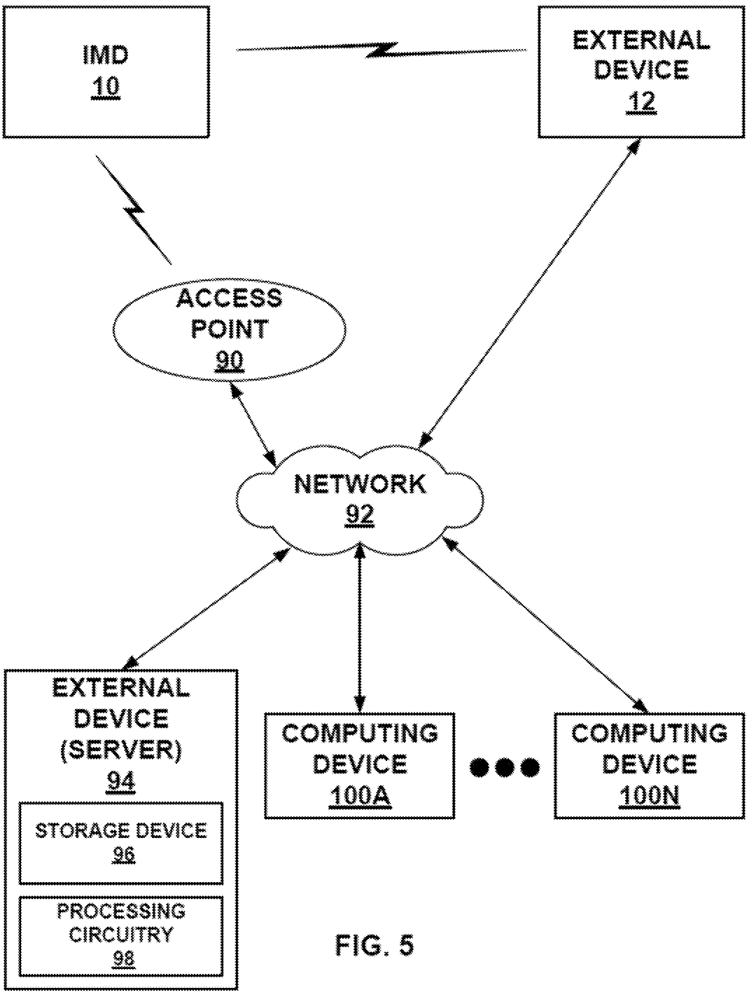

External device 12 may be used to configure operational parameters for IMD 10A or IMD 10B. External device 12 may be used to retrieve data from IMD 10A or IMD 10B. The retrieved data may include values of physiological parameters measured by IMD 10A or IMD 10B, indications of patient health episodes or other maladies detected by IMD 10A or IMD 10B, and physiological signals recorded by IMD 10A or IMD 10B. For example, external device 12 may retrieve physiological data recorded by IMD 10A or IMD 10B. As discussed in greater detail below with respect to FIG. 5, one or more remote computing devices may interact with IMD 10A or 10B in a manner similar to external device 12, e.g., to program IMD 10A or 10B and/or retrieve data from IMD 10A or 10B, via a network.

Processing circuitry of medical system 2, e.g., of IMD 10A, IMD 10B, external device 12, and/or of one or more other computing devices, may be configured to perform the example techniques of this disclosure for determining which sets of layers of a ML model to deactivate or activate to analyze sensed data.

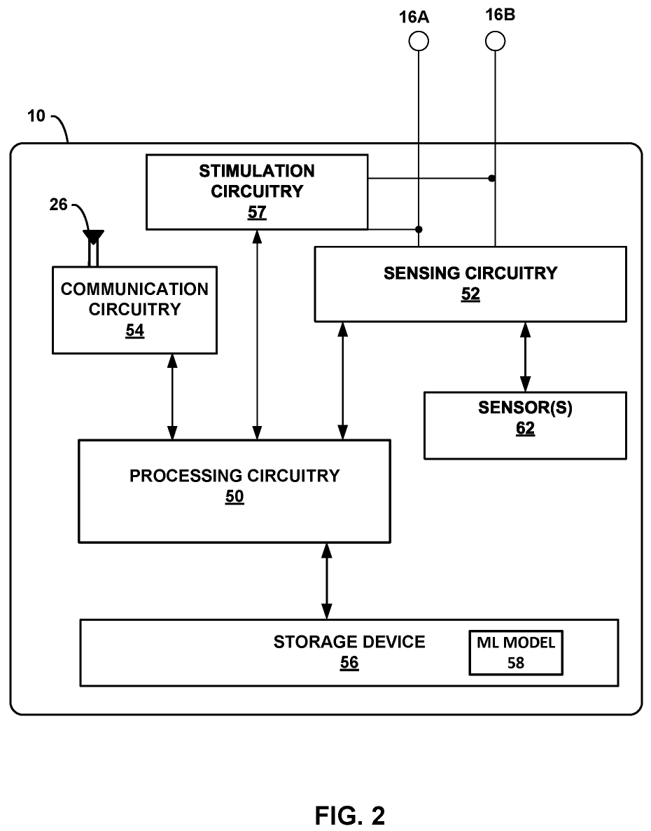

FIG. 2 is a functional block diagram illustrating an example configuration of IMD 10, which is example of IMD 10A of FIG. 1A or IMD 10B of FIG. 1B in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, stimulation circuitry 57, and sensors 62. In some examples, electrodes 16 may refer to at least one of the electrodes of leads 150. Although the illustrated example includes two electrodes 16, IMDs including or coupled to more than two electrodes 16 may implement the techniques of this disclosure in some examples. However, IMD 10 does not need to include all these components in some examples, or IMD 10 may include additional components in some examples. For example, IMD 10 may not include stimulation circuitry 57, in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may sense signals from electrodes 16 or sensors 62, e.g., to produce a cardiac EGM, EEG signal, ECAP signal, or other signal in order to facilitate monitoring one or more particular physiological signals of the patient. Stimulation circuitry 57 may be coupled to electrodes 16 and configured to cause electrical stimulation therapy to be delivered to patient 4 via one or more electrodes 16. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

Sensing circuitry 52 may also provide one or more digitized physiological signals to processing circuitry 50 for analysis, e.g., to determine which set of layers of ML model 58 to deactivate, and to apply physiological data to the activated layers of the ML model 58 to detect a presence of a physiological signal in sensed physiological data or determine a physiological parameter and/or health condition of the patient according to the techniques of this disclosure. In some examples, processing circuitry 50 may store the digitized physiological signals in storage device 56.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store one or more machine learning models 58, such as described herein. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include digitized cardiac EGMs, as examples.

In some examples, stimulation circuitry 57 is configured to generate and deliver electrical therapy to the patient. Stimulation circuitry 57 may include one or more pulse generators, capacitors, and/or other components capable of generating and/or storing energy to deliver as electrical stimulation therapy.

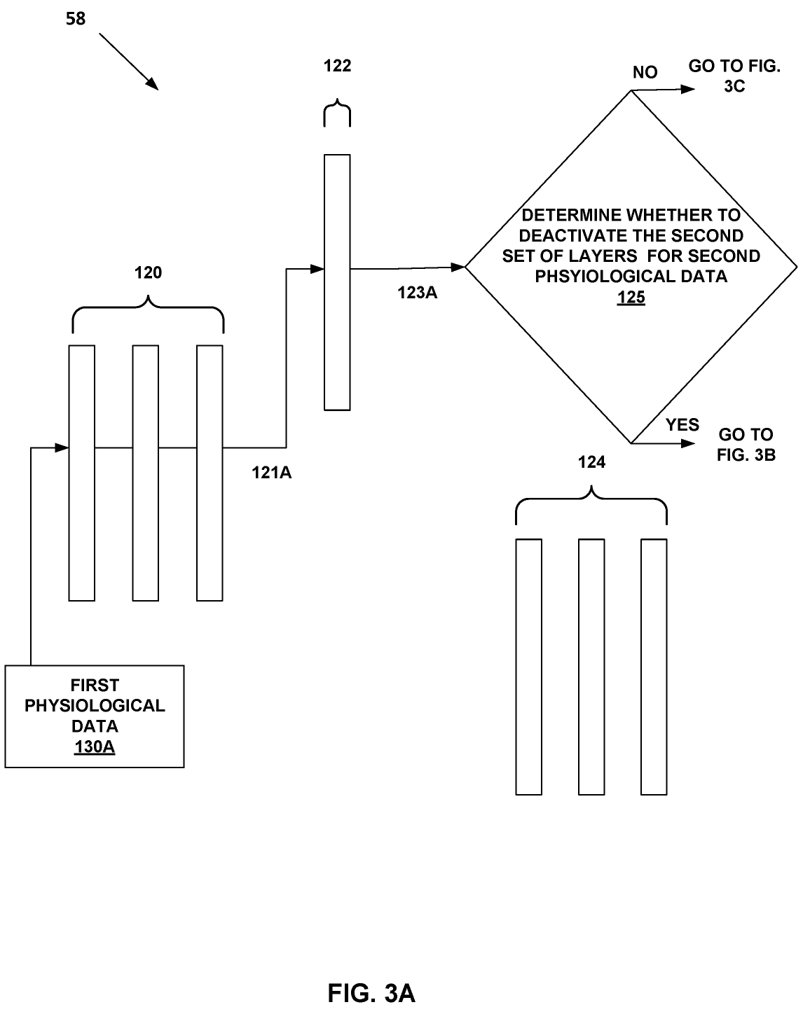

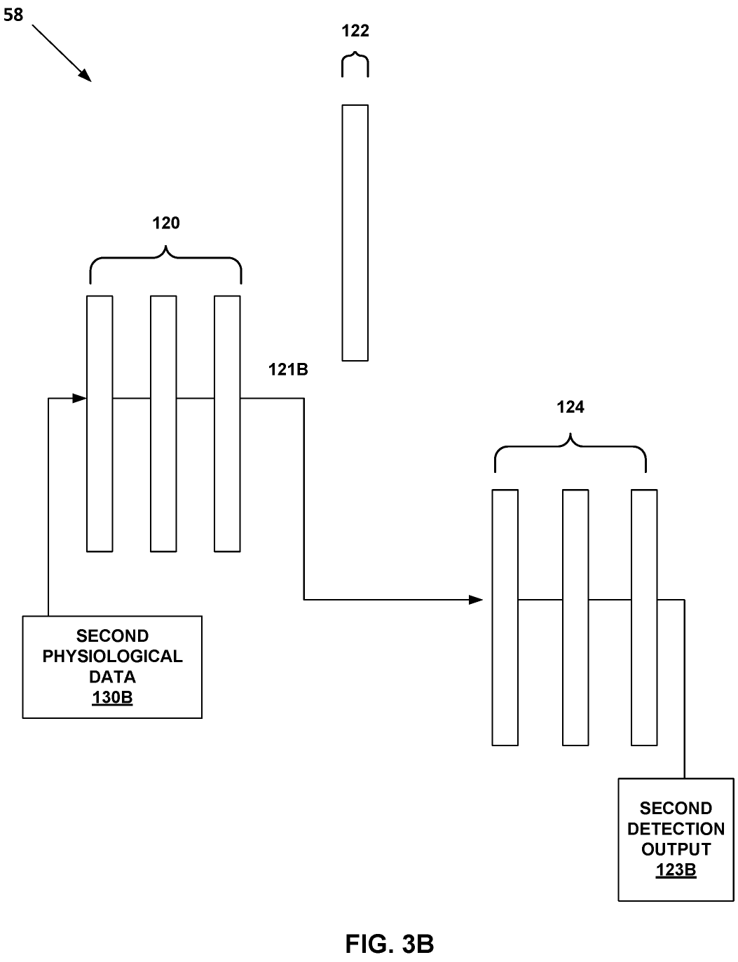

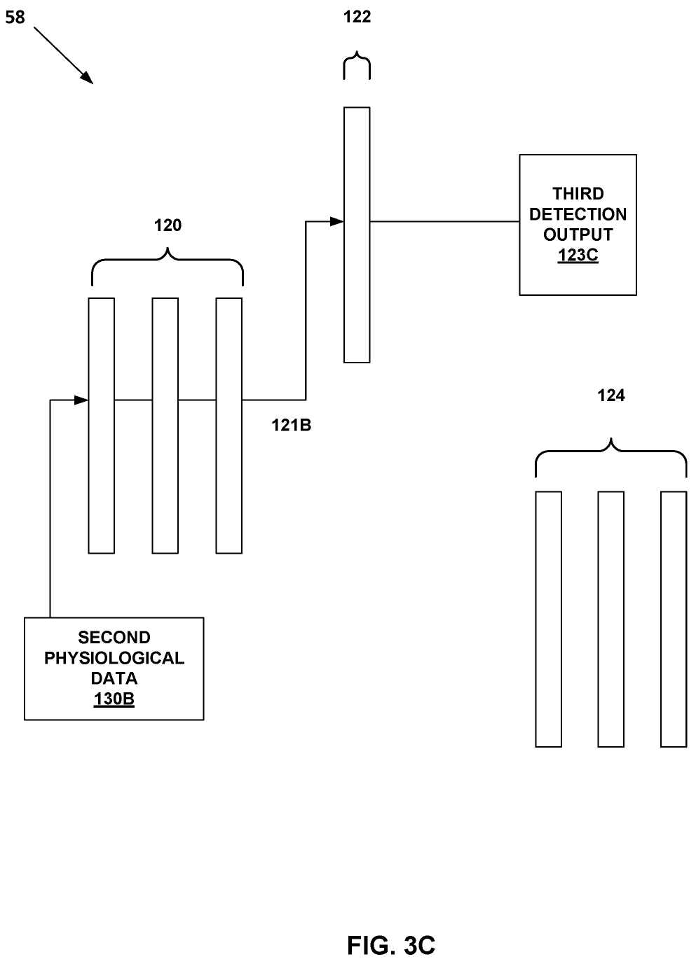

FIGS. 3A-3C are conceptual diagrams illustrating an example ML model 58 configured to determine which set of layers of ML model 58 to deactivate and to apply physiological data to the activated layers of the ML model 58 to detect a presence of a physiological signal in the physiological data or determine a physiological parameter and/or health condition of a patient according to the techniques of this disclosure. ML model 58 is an example of a deep learning model, or deep learning algorithm, trained to determine a physiological parameter and/or health condition based on physiological data of a sensed signal. One or more of IMD 10, external device 12, server 94, and/or computing device(s) 100 may train, store, and/or utilize ML model 58, but other devices may apply inputs associated with a particular patient to ML model 58 in other examples. As discussed above, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

Although the techniques for determining whether to deactivate particular sets of layers of ML model 58 and to apply physiological data to the activated layers of the ML model 58 to detect a presence of a particular physiological signal in the physiological data, determine a physiological parameter and/or determine health condition of a patient is described as being performed by processing circuitry 50 of IMD 10, such techniques may be performed, in whole or part, by processing circuitry of any one or more devices of system 2, such as processing circuitry 80 of external device 12, processing circuitry 98 of server 94, or processing circuitry of one or more computing devices 100.

As shown in the examples of FIGS. 3A-3C, ML model 58 may include three sets of layers, including a first set of layers 120, a second set of layers 122, and a third set of layers 124. In some examples, ML model 58 may include more than three sets of layers. FIGS. 3A-3C show the first set of layers 120 having three layers, the second set of layers 122 having one layer and the third set of layers 124 having three layers. In some examples, the first set of layers 120 may have more or less than three layers. In some examples, the second set of layers 122 may have more than one layer. In some examples, the third set of layers 124 may have more or less than three layers. In some examples, sensed physiological data, such as first physiological data 130A and second physiological data 130B, may be provided as input to ML model 58, such as at the first set of layers 120.

In some examples, a layer may include interconnected nodes (or neurons) that perform specific computations. In some examples, a layer may comprise a linear transformation or non-linear transformation of an input into the layer. In some examples, a layer may be an input layer that may be configured to receive sensed physiological data, such as first physiological data 130A and second physiological data 130B. In some examples, a layer may be a convolutional layer that may be configured to detect signal patters. In some examples, a layer may be a recurrent layer that may be configured to analyze temporal trends in the sensed physiological data. In some examples, a layer may be an output layer that is configured to determine and generate a respective output of the ML model based on the processed information of the earlier layers. In some examples, where a particular layer is positioned with respect to other layers may indicate what type of layer the particular layer will be.

For example, as shown in FIGS. 3A-3C, the first set of layers 120 may include an input layer and two hidden layers, the second set of layers 122 including an output layer, and the third set of layers 124 including two hidden layers and one output layer. However, the first set of layers 120, the second set of layers 122, and the third set of layers 124 may have more or less layers than indicated in the examples shown in FIGS. 3A-3C.

Although the techniques for determining which set of layers of ML model 58 to deactivate may refer to an ECAP signal as an example of the physiological signal, such techniques may be used for other physiological signals as well, such as EGM, ECG, EEG, EMG, EP, local field potential, signals that vary based on respiration, signals that vary based on blood pressure, signals that vary based on activity level, signals that vary based on posture, signals that vary based on sleep, signals that vary based on medication state, etc. In some examples, the second set of layers 122 may be a shallow output layer configured to determine whether physiological data includes an obvious particular signal. For example, IMD 10 may be configured to detect an ECAP signal, but the presence of an ECAP signal in the sensed physiological data may differ from patient to patient. In some patients, an ECAP signal may be easily apparent in the sensed physiological data and can be determined via a shallow output layer of a ML model, such as the second set of layers 122. In other patients, an ECAP signal may not be easily apparent in sensed physiological data and may require a more computationally expensive set of layers to determine whether an ECAP signal is present in the sensed physiological data in the particular patient. In patients in which an ECAP signal may be easily apparent in the sensed physiological data, the second set of layers 122 may be able to determine the presence of the ECAP signal in the sensed physiological data. In patients in which an ECAP signal may not be easily apparent in the sensed physiological data, the second set of layers 122 may be unable to determine the presence of the ECAP signal in the sensed physiological data. In some examples, the output of the second set of layers 122 for patients in which an ECAP signal may not be easily apparent in the sensed physiological data may be inconsequential to determining whether an ECAP signal is present in the sensed physiological data and continuing to apply extracted features from the first set of layers 120 to the second set of layers 122 may be computationally expensive while not providing significant help to determine whether an ECAP signal is present in the sensed physiological.

In accordance with techniques described herein, processing circuitry 50 may dynamically deactivate a set of layers in ML model 58 to save computational resources and improve efficiency of the ML model 58. In some examples, processing circuitry 50 may dynamically deactivate the second set of layers 122 to enable ML model 58 to detect whether a particular physiological signal, such as an ECAP, EEG, ECG, etc., is present in sensed physiological data to save computational resources and improve efficiency of ML model 58. For example, when ML model 58 is operating on IMD 10, computational resources and/or power may be limited due to size limitations of an IMD. An IMD 10 having a ML model 58, in accordance with techniques described herein, that is configured to operate with improved efficiency may enable the IMD 10 to have a greater lifespan and/or enable the IMD 10 to run the ML model 58 more often, which may help produce improved clinical results for a patient. In some examples, having one ML model 58 that dynamically deactivates particular sets of layers to determine whether a particular signal is detected in sensed physiological data instead of multiple separate ML models may save computational resources, such as memory, of an IMD 10.

In some examples, processing circuitry 50 may apply first physiological data 130A to the first set of layers 120 to generate a first base output 121A. In some examples, the first base output 121A may include one or more features extracted from the first physiological data 130A. Processing circuitry 50 may be configured to apply the first base output 121A to the second set of layers 122 to generate a first detection output 123A. In some examples, processing circuitry 50 may determine whether to deactivate the second set of layers 122 for second physiological data 130B based on the first detection output 123A (125). The second physiological data 130B being sensed after the first physiological data 130A. In some examples, processing circuitry 50 may determine whether the first detection output 123A satisfies a deactivation threshold. In some examples, the first detection output 123A satisfying the deactivation threshold may correspond to the first detection output 123A, which is output by the second set of layers 122, indicating a physiological signal, such as an ECAP signal, ECG signal, or EEG signal, is not detected in the first physiological data. In some examples, in response to a determination the first detection output 123A satisfies the deactivation threshold, processing circuitry 50 may determine to deactivate the second set of layers 122. In some examples, in response to a determination the first detection output 123A satisfies the deactivation threshold, processing circuitry 50 may additionally determine to activate the third set of layers 124.

In some examples, processing circuitry 50 may apply first physiological data 130A to the first set of layers 120, second set of layers 122, and the third set of layers 124 (e.g., fully activated ML model 58) to determine how many occasions an “obvious” physiological signal, such as an ECAP signal, is present in the first physiological data 130A. Processing circuitry 50 may then apply first physiological data 130A to the first set of layers 120 and the second set of layers 122 to determine (e.g., without using third set of layers 124) how many occasions an “obvious” physiological signal, such as an ECAP signal, is present in the first physiological data 130A. In some examples, processing circuitry 50 may compare the amount of “obvious” signals detected using a fully activated ML model 58 compared to the amount of “obvious” signals detected using the first set of layers 120 and the second set of layers 122 without using the third set of layers 124 to determine an “obvious” signal detection difference. In some examples, processing circuitry 50 may determine an amount of power consumption saved by not running the third set of layers 124, such as by multiplying the amount of “obvious” signals that did not need to be processed by the third set of layers 124 by the power consumption of running the third set of layers 124. In some examples, processing circuitry 50 may determine a penalty value of a power consumption cost of running the second set of layers 122. In some examples, processing circuitry 50 may compare the penalty value of running the second of layers 122 to the determined power savings of running the third set of layers 124 to determine whether the deactivation threshold is satisfied. For example, if the power savings of not running the third set of layers 124 does not outweigh the penalty of running the second set of layers 122, processing circuitry 50 may deactivate the second set of layers 122. In some examples, if the power savings of not running the third set of layers 124 does outweigh the penalty of running the second set of layers 122, processing circuitry 50 may keep the second set of layers 122 activated.

Processing circuitry 50 is configured to apply second physiological data 130B to the first set of layers 120 to generate a second base output 121B. In some examples, second base output 121B may include one or more features extracted of the second physiological data 130B. As shown in the example of FIG. 3B, in response to a determination to deactivate the second set of layers 122, processing circuitry 50 is configured to apply the second base output 121B to the third set of layers 124 to generate a second output detection 123B. In some examples, executing the second set of layers 122 requires less computational resources than executing the third set of layers 124, but executing both the second set of layers 122 and the third set of layers 124 requires more computational resource than executing the third set of layers 124 while the second set of layers 122 is deactivated. In some examples, dynamically deactivating the second set of layers 122 based on the first detection output 123A may save computational resources and improve efficiency of ML model 58, such as when the ML model 58 is operating on an IMD 10, when applying second physiological data 130B to ML model 58 to determine a second detection output 123B.

In some examples, in response to a determination the first detection output 123A does not satisfy the deactivation threshold (e.g., a physiological signal, such as an ECAP, is easily detected in the first detection output 123A of the second set of layers), processing circuitry 50 may determine to maintain the activation status of the second set of layers 122 or activate the second set of layers 122 or keep the second set of layers 122 active.

In some examples, as shown in the example of FIG. 3C, in response to a determination to maintain the activation status of the second set of layers 122 or activate the second set of layers 122, processing circuitry 50 is configured to apply the second base output 121B to the second set of layers 122 to generate a third detection output detection 123C. In some examples, as shown in the example of FIG. 3C, in response to a determination to activate the second set of layers 122, processing circuitry 50 may additionally be configured to deactivate the third set of layers 124.

In some examples, processing circuitry 50 may be configured to determine whether to deactivate the second set of layers 122 for physiological data continuously or periodically. For example, after a determination to deactivate the second set of layers 122, processing circuitry 50 may be configured to apply newly generated physiological data, based on new signals sensed by IMD 10 a period of time after the signals sensed corresponding to the second physiological data 130B, to determine whether to deactivate the second set of layers 122 for newly generated physiological data. In some examples, the period of time may be up to 5 seconds, up to 10 seconds, up to 30 seconds, up to 1 minute, up to 30 minutes, up to 1 hour, or up to other amounts of time. In some examples, a change of one or more features of the sensed signals may trigger processing circuitry 50 to apply physiological data based on newly sensed signals to the first set of layers 120 and the second set of layers 122 of ML model 58, as described herein, to determine whether to deactivate the second set of layers 122 for additionally generated physiological data.

In some examples, stimulation circuitry 57 may be configured to generate electrical stimulation therapy and processing circuitry 50 may be configured to adjust one or more parameters that define the electrical stimulation therapy 57 based on at least one of the first detection output 123A or the second detection output 123B. In some examples, processing circuitry 50 may be configured to adjust one or more parameters that define the electrical stimulation therapy 57 based on at least one of the first detection output 123A, the second detection output 123B, or the third detection output 123C.

The techniques for determining whether to deactivate particular sets of layers of ML model 58 and to apply physiological data to the activated layers of the ML model 58 to detect a presence of a particular physiological signal in the physiological data, determine a physiological parameter and/or determine health condition of a patient is described as being performed by processing circuitry 50 of IMD 10. However, such techniques may be performed, in whole or part, by processing circuitry of any one or more devices of system 2, such as processing circuitry 80 of external device 12, processing circuitry 98 of server 94, or processing circuitry of one or more computing devices 100.

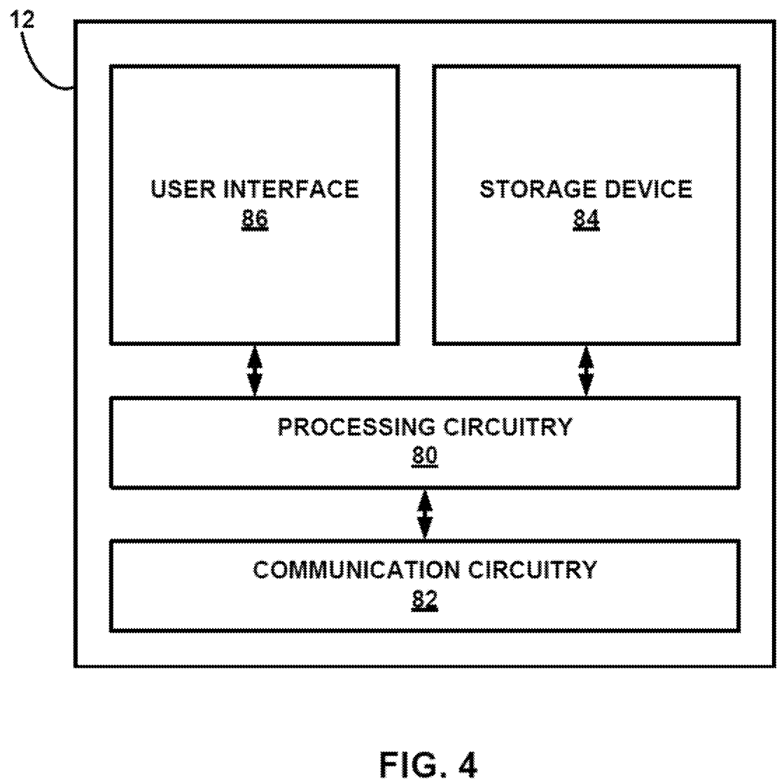

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., digitized physiological signals) to external device 12. In turn, external device 12 may receive the collected data from IMD 10 and store the collected data in storage device 84. In some examples, storage device 84 may store a ML model. Processing circuitry 80 may implement any of the techniques described herein to determine whether to deactivate a set of layers of a ML model.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 5 is a block diagram illustrating an example system that includes an access point 90, a network 92, external computing devices, such as a server 94, and one or more other computing devices 100A–100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as physiological data, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94. or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. 5 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 100 may implement any of the techniques described herein to determine whether to deactivate a set of layers of a ML model.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

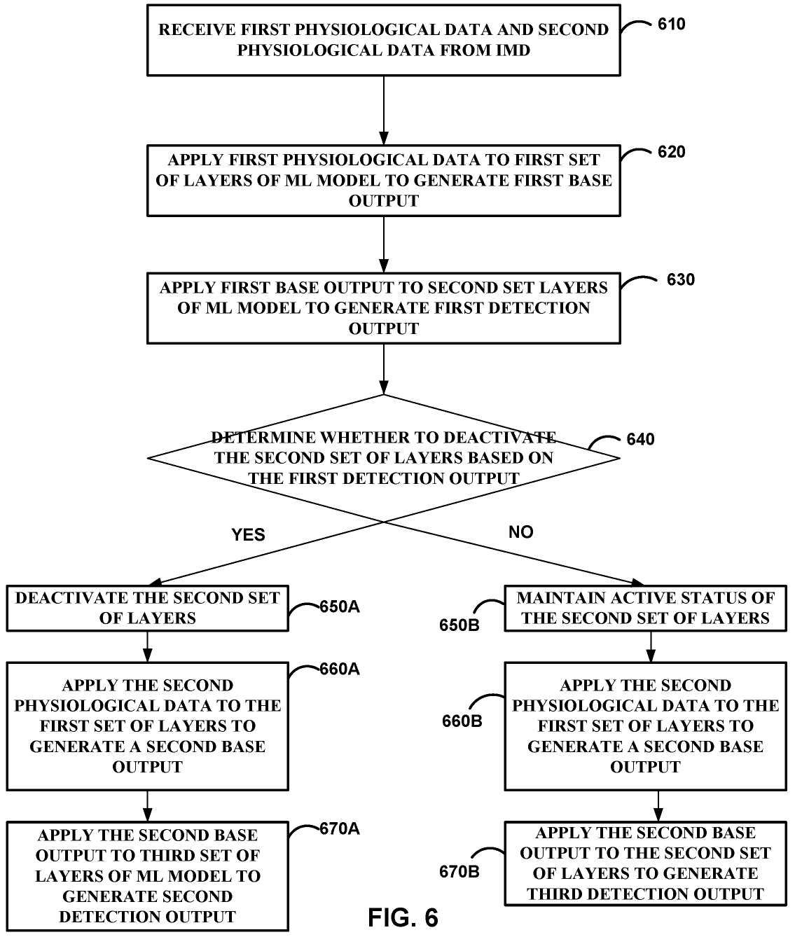

FIG. 6 is a flow diagram illustrating an example technique for medical system 2. As indicated by FIG. 6, processing circuitry 50 may be configured to receive first physiological data and second physiological data from an IMD 10 (610). In some examples, the first physiological data and the second physiological data may include and/or be based on signal(s) sensed by one or more sensors of IMD 10. The second physiological data may be sensed after the first physiological data. Processing circuitry 50 may apply the first physiological data to a first set of layers 120 of ML model 58 to generate a first base output (620). In some examples, the first base output may include features of the first physiological data extracted by the first set of layers 120. In some examples, ML model 58 may be stored in storage medium 56 of IMD 10. Processing circuitry 50 may apply the first base output to a second set of layers 122 of ML model 58 to generate a first detection output (630). Processing circuitry 50 may determine whether to deactivate the second set of layers 122 of ML model 58 based on the first detection output (640). In some examples, processing circuitry 50 may determine to deactivate the second set of layers 122 of ML model 58 based on the first detection output satisfying a deactivation threshold. In some examples, the first detection output satisfying the deactivation threshold may correspond to the first detection output, which is output by the second set of layers 122 of the ML model 58, indicating a particular physiological signal, such as an ECAP signal, ECG signal, or EEG signal, is not detected in the first physiological data 130A (e.g., the second set of layers 122 of the ML model 58 was unable to identify a satisfactory desired particular signal, such as an ECAP, in the physiological data 130A).

In some examples, processing circuitry 50 may determine to maintain the activate status of the second set of layers 122 of ML model 58 based on the first detection output not satisfying a deactivation threshold. In some examples, the first detection output not satisfying the deactivation threshold may correspond to the first detection output, which is output by the second set of layers of the ML model, indicating a physiological signal, such as an ECAP signal, ECG signal, or EEG signal, is detected in the first physiological data (e.g., the second set of layers of the ML model was able to identify a satisfactory desired particular signal, such as an ECAP, in the physiological data).

In response to a determination to deactivate the second set of layers 122 of ML model 58 (“YES” branch of block 640), processing circuitry 50 may deactivate the second set of layers (650A), apply the second physiological data 130B to the first set of layers 120 to generate a second base output (660A), and apply the second base output to a third set of layers 124 of ML model 58 to generate a second detection output (670A).

In response to a determination to not deactivate the second set of layers 122 of ML model 58 (“NO” branch of block 640), processing circuitry 50 may maintain active status of the second set of layers (650B), apply the second physiological data 130B to the first set of layers to generate a second base output (660B), and apply the second base output to the second set of layers 124 of ML model 58 to generate a third detection output (670B).

In some examples, processing circuitry 50 may be configured to determine whether to deactivate the second set of layers 122 for physiological data continuously or periodically. In some examples, after a determination to deactivate the second set of layers 122, processing circuitry 50 may be configured to apply newly generated physiological data, based on newly signals sensed by IMD 10 a period of time after the signals sensed corresponding to the second physiological data 130B, to determine whether to deactivate the second set of layers 122 for newly generated physiological data. In some examples, the period of time may be up to 5 seconds, up to 10 seconds, up to 30 seconds, up to 1 minute, up to 30 minutes, up to 1 hour, or up to other amounts of time. In some examples, a change of one or more features of the sensed signals may trigger processing circuitry 50 to apply physiological data based on newly sensed signals to the first set of layers 120 and the second set of layers 122 of ML model 58, as described herein, to determine whether to deactivate the second set of layers 122 for newly generated physiological data based on newly sensed signals.

In some examples, stimulation circuitry 57 may be configured to generate electrical stimulation therapy and processing circuitry 50 may be configured to adjust one or more parameters that define the electrical stimulation therapy 57 based on at least one of the first detection output or the second detection output. In some examples, processing circuitry 50 may be configured to adjust one or more parameters that define the electrical stimulation therapy 57 based on at least one of the first detection output, the second detection output, or the third detection output.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following examples are illustrative of the techniques described herein.

Example 1: An implantable medical device includes sensing circuitry configured to generate first physiological data representative of first physiological signals of a patient and second physiological data representative of second physiological signals of the patient, the second physiological signals being sensed after the first physiological signals; a storage medium configured to store a machine learning (ML) model that includes a plurality of layers, the plurality of layers comprising a first set of layers, a second set of layers, and a third set of layers; and processing circuitry configured to receive the first physiological data and the second physiological data from the sensing circuitry; apply the first physiological data to the first set of layers to generate a first base output; apply the first base output to the second set of layers to generate a first detection output; determine, based on the first detection output, to deactivate the second set of layers for the second physiological data; in response to the determination to deactivate the second set of layers: deactivate the second set of layers; apply the second physiological data to the first set of layers to generate a second base output; and apply the second base output to the third set of layers to generate a second detection output.

Example 2: The system of example 1, wherein the first base output includes one or more features extracted from the first physiological data and the second base output includes one or more features extracted from the second physiological data.

Example 3: The system of any of examples 1-2, wherein the processing circuitry is further configured to: determine that the first detection output satisfies a deactivation threshold; and in response to the determination the first detection output satisfies the deactivation threshold, deactivate the second set of layers.

Example 4: The system of example 3, wherein the processing circuitry is further configured to: in response to the determination the first detection output satisfies the deactivation threshold, activate the third set of layers.

Example 5: The system of any of examples 1-2, wherein in response to a determination to not deactivate the second set of layers, the processing circuitry is further configured to: maintain active status of the second set of layers; apply the second physiological data to the first set of layers to generate the second base output; and apply the second base output to the second set of layers to generate a third detection output.

Example 6: The system of example 5, wherein the processing circuitry is further configured to: determine that the first detection output does not satisfy a deactivation threshold; in response to the determination that the first detection output does not satisfy the deactivation threshold, maintain the active status of the second set of layers.

Example 7: The system of example 6, wherein the processing circuitry is configured to: in response to the determination that the first detection output does not satisfy the deactivation threshold, deactivate the third set of layers.

Example 8: The system of any of examples 1-7, wherein the second set of layers of the ML model is less computationally complex than the third set of layers of the ML model.

Example 9: The system of any of examples 1-8, further comprising stimulation circuitry configured to generate electrical stimulation therapy, and wherein the processing circuitry is configured to adjust, based on at least one of the first detection output or the second detection output, one or more parameters that define the electrical stimulation therapy.

Example 10: A method includes generating, by sensing circuitry of an implantable medical device (IMD), first physiological data representative of first physiological signals of a patient and second physiological data representative of second physiological signals of the patient; applying, by processing circuitry of the IMD, the first physiological data to a first set of layers of a machine learning (ML) model to generate a first base output; applying, by the processing circuitry, the first base output to a second set of layers of the ML model to generate a first detection output; determining, by the processing circuitry and based on the first detection output, to deactivate the second set of layers for the second physiological data based on the first detection output; in response to the determination to deactivate the second set of layers: deactivating, by the processing circuitry, the second set of layers; applying, by the processing circuitry, the second physiological data to the first set of layers to generate a second base output; and applying, by the processing circuitry, the second base output to a third set of layers of the ML model to generate a second detection output.

Example 11: The method of example 10, further includes sensing, by the IMD, the first physiological signal and the second physiological signals, wherein the second physiological signals is sensed after the first physiological signals.

Example 12: The method of any of examples 10-11, wherein the first base output includes one or more features extracted from the first physiological data and the second base output includes one or more features extracted from the second physiological data.

Example 13: The method of any of examples 10-12, further includes determining, by the processing circuitry, that the first detection output satisfies a deactivation threshold; and in response to the determination the first detection output satisfies the deactivation threshold, deactivating, by the processing circuitry, the second set of layers.

Example 14: The method of example 13, further includes in response to the determination the first detection output satisfies the deactivation threshold, activating, by the processing circuitry, the third set of layers.

Example 15: The method of any of examples 10-12, further includes in response to a determination to not deactivate the second set of layers, maintaining, by the processing circuitry, active status of the second set of layers; applying, by the processing circuitry, the second physiological data to the first set of layers to generate the second base output; and applying, by the processing circuitry, the second base output to the second set of layers to generate a third detection output.

Example 16: The method of example 15, further includes determining, by the processing circuitry, that the first detection output does not satisfy a deactivation threshold; and in response to the determination that the first detection output does not satisfy the deactivation threshold, maintaining, by the processing circuitry, the active status of the second set of layers.

Example 17: The method of example 16, further includes in response to the determination that the first detection output does not satisfy the deactivation threshold, deactivating, by the processing circuitry, the third set of layers.

Example 18: The method of any of examples 10-17, wherein executing the second set of layers of the ML model is less computationally complex than the third set of layers of the ML model.

Example 19: The method of any of examples 10-18, further includes generating, by stimulation circuitry of the IMD, electrical stimulation therapy; and adjusting, by the processing circuitry and based on at least of the first detection output or the second detection output, one or more parameters that define the electrical stimulation therapy.

Example 20: A computer-readable medium comprising instructions that, when executed, causes processing circuitry of a medical system to perform the method of any of examples 10-19.

Various examples have been described. These and other examples are within the scope of the following claims.

What is claimed is:

1. An implantable medical device, the device comprising:
sensing circuitry configured to generate first physiological data representative of first physiological signals of a patient and second physiological data representative of second physiological signals of the patient, the second physiological signals being sensed after the first physiological signals;
a storage medium configured to store a machine learning (ML) model comprising a plurality of layers, the plurality of layers comprising a first set of layers, a second set of layers, and a third set of layers; and
processing circuitry configured to:
receive the first physiological data and the second physiological data from the sensing circuitry;
apply the first physiological data to the first set of layers to generate a first base output;
apply the first base output to the second set of layers to generate a first detection output;
determine, based on the first detection output, to deactivate the second set of layers for the second physiological data; and
in response to the determination to deactivate the second set of layers:
deactivate the second set of layers;
apply the second physiological data to the first set of layers to generate a second base output; and
apply the second base output to the third set of layers to generate a second detection output.

2. The device of claim 1, wherein the first base output includes one or more features extracted from the first physiological data and the second base output includes one or more features extracted from the second physiological data.

3. The device of claim 1, wherein the processing circuitry is further configured to:
determine that the first detection output satisfies a deactivation threshold; and
in response to the determination the first detection output satisfies the deactivation threshold, deactivate the second set of layers.

4. The device of claim 3, wherein the processing circuitry is further configured to:
in response to the determination the first detection output satisfies the deactivation threshold, activate the third set of layers.

5. The device of claim 1, wherein in response to a determination to not deactivate the second set of layers, the processing circuitry is further configured to:
maintain active status of the second set of layers;
apply the second physiological data to the first set of layers to generate the second base output; and
apply the second base output to the second set of layers to generate a third detection output.

6. The device of claim 5, wherein the processing circuitry is further configured to:
determine that the first detection output does not satisfy a deactivation threshold;
in response to the determination that the first detection output does not satisfy the deactivation threshold, maintain the active status of the second set of layers.

7. The device of claim 6, wherein the processing circuitry is configured to:
in response to the determination that the first detection output does not satisfy the deactivation threshold, deactivate the third set of layers.

8. The device of claim 1, wherein the second set of layers of the ML model is less computationally complex than the third set of layers of the ML model.

9. The device of claim 1, further comprising stimulation circuitry configured to generate electrical stimulation therapy, and wherein the processing circuitry is configured to adjust, based on at least one of the first detection output or the second detection output, one or more parameters that define the electrical stimulation therapy.

10. A method comprising:
generating, by sensing circuitry of an implantable medical device (IMD), first physiological data representative of first physiological signals of a patient and second physiological data representative of second physiological signals of the patient;
applying, by processing circuitry of the IMD, the first physiological data to a first set of layers of a machine learning (ML) model to generate a first base output;
applying, by the processing circuitry, the first base output to a second set of layers of the ML model to generate a first detection output;
determining, by the processing circuitry and based on the first detection output, to deactivate the second set of layers for the second physiological data based on the first detection output; and
in response to the determination to deactivate the second set of layers:
deactivating, by the processing circuitry, the second set of layers;
applying, by the processing circuitry, the second physiological data to the first set of layers to generate a second base output; and
applying, by the processing circuitry, the second base output to a third set of layers of the ML model to generate a second detection output.

11. The method of claim 10, further comprising:
sensing, by the IMD, the first physiological signal and the second physiological signals, wherein the second physiological signals is sensed after the first physiological signals.

12. The method of claim 10, wherein the first base output includes one or more features extracted from the first physiological data and the second base output includes one or more features extracted from the second physiological data.

13. The method of claim 10, further comprising:
determining, by the processing circuitry, that the first detection output satisfies a deactivation threshold; and
in response to the determination the first detection output satisfies the deactivation threshold, deactivating, by the processing circuitry, the second set of layers.

14. The method of claim 13, further comprising:
in response to the determination the first detection output satisfies the deactivation threshold, activating, by the processing circuitry, the third set of layers.

15. The method of claim 10, further comprising:
in response to a determination to not deactivate the second set of layers,
maintaining, by the processing circuitry, active status of the second set of layers;
applying, by the processing circuitry, the second physiological data to the first set of layers to generate the second base output; and
applying, by the processing circuitry, the second base output to the second set of layers to generate a third detection output.

16. The method of claim 15, further comprising:
determining, by the processing circuitry, that the first detection output does not satisfy a deactivation threshold; and
in response to the determination that the first detection output does not satisfy the deactivation threshold, maintaining, by the processing circuitry, the active status of the second set of layers.

17. The method of claim 16, further comprising:
in response to the determination that the first detection output does not satisfy the deactivation threshold, deactivating, by the processing circuitry, the third set of layers.

18. The method of claim 10, wherein executing the second set of layers of the ML model is less computationally complex than the third set of layers of the ML model.

19. The method of claim 10, further comprising:
generating, by stimulation circuitry of the IMD, electrical stimulation therapy; and
adjusting, by the processing circuitry and based on at least of the first detection output or the second detection output, one or more parameters that define the electrical stimulation therapy.

20. A computer-readable medium comprising instructions that, when executed, causes processing circuitry of a medical system to:
generate first physiological data representative of first physiological signals of a patient and second physiological data representative of second physiological signals of the patient;
apply the first physiological data to a first set of layers of a machine learning (ML) model to generate a first base output;
apply the first base output to a second set of layers of the ML model to generate a first detection output;
determine, based on the first detection output, to deactivate the second set of layers for the second physiological data based on the first detection output; and
in response to the determination to deactivate the second set of layers:
deactivate the second set of layers;
apply the second physiological data to the first set of layers to generate a second base output; and
applying the second base output to a third set of layers of the ML model to generate a second detection output.
